(19) United States
(12) Patent Application Publication
(10) Pub. No.: US 2016/0051178 A1
(43) Pub. Date: Feb. 25, 2016

(54) SYSTEM AND METHOD FOR CALCULATING BRAIN VOLUME

(71) Applicant: Virginia Institute of Neuropsychiatry, Midlothian, VA (US)

(72) Inventor: David E. Ross, Midlothian, VA (US)

(21) Appl. No.: 14/464,657

(22) Filed: Aug. 20, 2014

(51) Int. Cl. A61B 5/00; A61B 5/055; A61B 5/107

(52) U.S. Cl. CPC A61B 5/4064; A61B 5/1073; A61B 5/055

(57) ABSTRACT

A system and method provides for calculating brain volume in a patient. The method and system includes receiving current intracranial volume data of a patient after a brain injury event and electronically calculating a current brain volume of the patient based on the current brain volume data. The method and system therein electronically calculates a prior brain volume based at least on the current brain volume data and prior age of the patient, the prior brain volume and age being at an earlier point in time, such as prior to a brain injury. The method and system further determines a brain volume change value based on a comparison of the current brain volume and the prior brain volume.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD OF INVENTION

The disclosed technology relates generally to brain volume estimation and more specifically to estimating a patient's brain volume at a time prior to the occurrence of a brain injury.

### BACKGROUND

Brain injuries cause a change in brain volume. The change in brain volume can correspond to a host of physical and mental issues. While the human brain changes in volume over a normal lifetime, deviations from normal or average brain volume adjustments are important indicators for not only diagnosing brain injury events, but also improving medical treatment.

There are generally known techniques for measuring brain volume based on a scan or other type of measurement. For example, one common technique is the utilization of existing software applied to a Magnetic Resonance Imaging (MRI) scan of a patient's brain. Determining current brain volume (i.e. based on one point in time) has limited value in cases where it is important to know or quantify a change in brain volume of the patient over time (e.g. based on two points in time) to place the brain volume measurement into a proper perspective.

For example, as the brain injury causes a change in brain volume, it is important to know the patient's brain volume prior to the brain injury, so that the pre-injury volume can be compared to the post-injury volume. Unfortunately but not unexpectedly, in most cases, there is no prior brain volume information (e.g. a prior MRI scan) because the injury was unplanned. In these cases, it would be useful to have a method for estimating pre-injury brain volume. However, the current methods for estimating earlier brain volume are very limited. There are only two such previously developed methods, and both are so limited that they almost never are useful in patients who have suffered a brain injury

One existing technique for estimating brain volume at an earlier point in life is described in “Cerebral Volume Loss, Cognitive Deficit, and Neuropsychological Performance: Comparative Measures of Brain Atrophy: II. Traumatic Brain Injury” by Tate, D. F., et al. published in the Journal of International Neuropsychological Society, 2011 (“Tate”). Tate expands upon “MR-Based Brain and Cerebrospinal Fluid Measurement after Traumatic Brain Injury: Correlation with Neuropsychological Outcome” by Blatter, Duane D., et al. published in the American Journal of Neuroradiology, January 1997 (“Blatter”).

Blatter and other researchers have found that during childhood and adolescence, the human brain grows rapidly, reaching maximal volume during adolescence (on average at age 13). The skull grows to be just big enough to cover the brain. During later life, typically the 30s and older, the brain begins to atrophy slowly. During later life, especially the 60s and older, the brain begins to atrophy at a more rapid rate. Later in life, despite the brain's atrophy, skull volume does not change. In other words, intracranial volume does not change during adulthood. Tate develop an equation using intracranial volume, measured later in life, to predict maximal brain volume attained earlier in life, where the patient does not have an intervening brain injury event in between.

The article “Human Brain Changes Across the Life Span: A Review of 56 Longitudinal Magnetic Resonance Imaging Studies” by Hedman, Anne, et al, Human Brain Mapping, 33: 1987-2002 (2012) (“Hedman”) is a meta-analysis of various studies in normal human subjects. Hedman used curve-fitting techniques to develop models that predicted brain growth/atrophy across the life span, as illustrated in FIG. 4. The Hedman method is useful for estimating normal change in brain volume, e.g. of an individual not having a brain injury. Unlike Tate, the Hedman method is useful for estimating brain volume change across the life span. However, the primary limitation of Hedman is that, while the change in brain volume over a period of time can be estimated based on an individual's age, the change in brain volume does not apply and cannot be applied to brain volume in subjects who are not normal, e.g. subjects who have a brain injury within that time period.

Therefore, in cases where pre-injury brain volume measurements or MRI scans are unavailable, prior brain volume estimation techniques—including Tate, Blatter and Hedman—fail to account for estimating pre-injury brain volume across the life span in subjects with injury or disease, e.g. patients who suffered a brain injury. The method of Tate only estimated brain volume at a single point in the lifespan, namely during early life when brain volume was maximal. The growth/atrophy curves of Hedman provide an excellent road map for predicting brain volume in normal subjects during the lifespan, but by themselves they do not provide a method for estimating brain volume in abnormal subjects, that is, patients with brain injury or disease.

As such, there exists a need for a system and method for using post-injury brain/head scan data to estimate pre-injury brain volume of a brain injury patient of any age.

### BRIEF DESCRIPTION

The present computerized method and system integrates the methods of Tate and Hedman in order to provide for estimating brain volume in a patient of any age before a brain injury or brain injury event. The method and system includes obtaining current brain and intracranial volume data of a patient after a brain injury and electronically calculating, i.e. measuring, current brain and intracranial volume of the patient. The method and system therein electronically estimates/calculates pre-injury brain volume based at least on the current intracranial volume data and age of the subject before injury.

The method and system further determine a brain volume change value based on a comparison of the current brain volume and the pre-injury brain volume. Whereupon, the present method and system therein provide for the determination of a patient's brain volume at a point earlier in time, i.e. prior to a brain injury event based at least on brain scan data taken after the brain injury event and the age of the patient before injury.

### BRIEF DESCRIPTION OF THE DRAWINGS

FIG. 1 illustrates a block diagram of a computer processing system providing for the calculating brain volume.

FIG. 2 illustrates a flow diagram of one embodiment of a computerized method for calculating brain volume.

FIG. 3 illustrates a representation of a brain segmentation illustrating brain regions as part of the brain scan data usable for calculating brain volume.

FIG. 4 illustrates a graphical depiction of the meta-analysis of Hedman, which determined the relationship between age and rate of whole brain volume change across the life span.

FIG. 5 illustrates a graphical representation of an integrated model for estimating whole brain parenchymal volume.

FIG. 6 is similar to FIG. 5, except it utilizes starting data from a plurality of normal control subjects instead of Hedman's starting data.

FIG. 7 is similar to FIG. 6, except that whole brain volume measured as a percentage of intracranial volume is added on the right-hand axis.

FIG. 8 is similar to FIG. 7, except that whole brain volume measured in cc is removed from the left-hand axis.

FIG. 9 is similar to FIG. 8, except that data for a single subject (RG) is overlaid on the group data.

FIG. 10 is similar to FIG. 9 except that the right-hand axis (% ICV) was removed and the left-hand axis (WBP volume measured in cc) was replaced, resulting in a shift of subject RG's data above the line for the normal control group because he had a larger ICV than the mean of the normal control group

FIG. 11 graphically shows the high correlation between WBP volume measured vs. estimated, supporting the reliability of the brain volume estimation method proposed herein.

FIG. 12 is similar to FIG. 9 except that subject RG's data are removed after age 30 because he had a brain injury at age 30, making estimates of brain volume after that age unreliable.

FIG. 13 illustrates a block diagram of a processing system allowing for receipt of scan data and calculating brain volume.

FIG. 14 illustrates a flow diagram of another embodiment of a computerized method for calculating brain volume.

FIG. 15 illustrates a graphical representation of cerebral white matter volume change occurring after a brain injury.

FIG. 16 illustrates a graphical representation of lower brain regions (SCN+IFT) volume changes after a brain injury.

FIG. 17 graphically summarizes the changes in brain volume in our sample of 26 patients with traumatic brain injury compared to our 20 normal control subjects.

A better understanding of the disclosed technology will be obtained from the following detailed description of the preferred embodiments taken in conjunction with the drawings and the attached claims.

### DETAILED DESCRIPTION

Embodiments of the disclosed technology comprise processing systems and methods for calculating a brain volume based on intracranial volume data, where the patient has suffered a brain injury event.

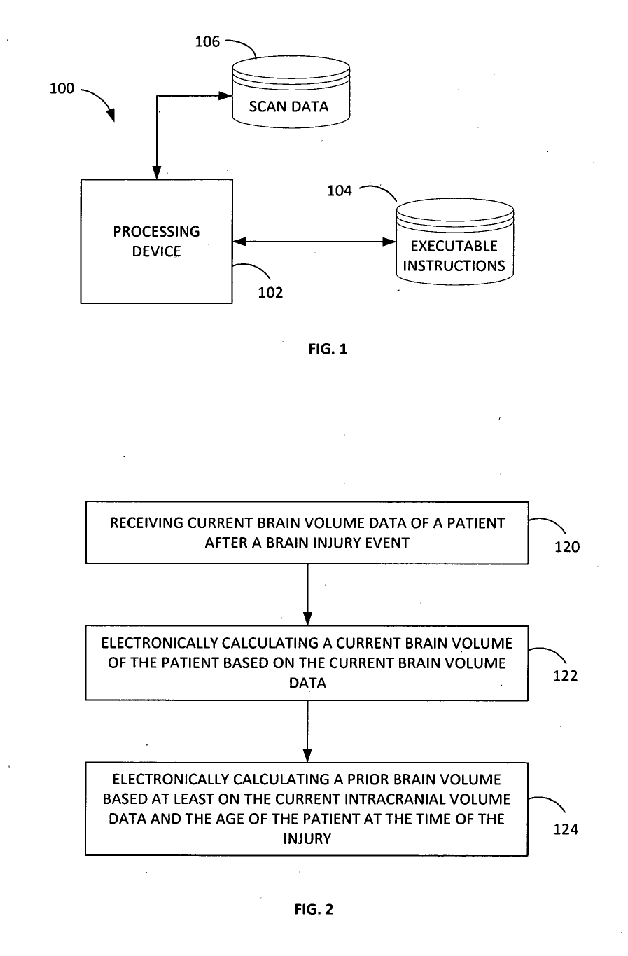

FIG. 1 illustrates one exemplary embodiment of a computing system 100 as described herein. The computing system provides for computing operations including the computerized calculating patient brain volume. The system 100 includes a processing device 102, executable instructions 104 stored in a computer readable medium and scan data 106.

The processing device 102 may be one or more processing devices operative to perform computations as described herein. The processing device may be, in one embodiment, a stand-alone processing device performing the processing operations in a single processing environment. In another embodiment the processing device may be any number of networked or interconnected processing devices performing processing operations in a networked or communication network. The processing device 102 includes networking and communication functionality as recognized by one skilled in the art, further discussions of known functionality being omitted for brevity purposes only.

The computer readable medium may be any suitable tangible medium operative to or capable of storing the executable instructions 104. The instructions 104 provide for instructing the processing device 102 to perform specific or various processing operations in response thereto. The executable instructions may be disposed in an internal storage medium to the processing device, or may be in a distributed processing environment such that the device 102 is operative to perform functionality as described herein.

The scan data 106 may be any suitable data relating to a patient including brain scan data 106. As described in further detail below, the brain scan data 106 may include scan data acquired from a magnetic resonance imaging (MRI) scan, which may be done locally or externally. The scan data 106 is not limited to MRI data, but rather any suitable data usable for determining a brain volume may be within the scan data 106 used herein.

For the sake of brevity, one embodiment of the operations of the processing device is described with respect to the steps of the flowchart of FIG. 2. FIG. 2 illustrates the steps of a method for calculating a brain volume of a patient prior to a brain injury event.

As used herein, a brain injury event, or brain injury, generally relates to any event occurrence having an adverse affect on a patient's brain. For example, a traumatic brain injury (TBI) is a type of a brain injury event. A TBI may include a concussion or series of concussions, such as arising from an accident, a fall or stumble, a sports-related concussion, by way of example. A degenerative disease or ailment is also a type of brain injury event, such as Alzheimer's disease by way of example. A brain injury or brain injury event may also include high stress or other environmental factors, such as military engagements or other high-stress scenarios where the patient suffers from posttraumatic stress disorder. As listed herein, the above are exemplary listings of brain injury events and not an exhaustive or limiting list, whereby the brain injury event may include any event having an adverse affect to the patient's brain, as recognized by one skilled in the art.

As described above in the background section, prior techniques exist for estimating a patient's prior brain volume, but such techniques fail to account for the occurrence of a brain injury event. Wherein, in the flowchart of FIG. 2, step 120 provides for receiving current brain volume data of a patient after a brain injury event. With respect to FIG. 1, the processing device 102 receives the scan data 106 from a storage location, wherein the scan data 106 represents brain measurement data, as well as other patient values.

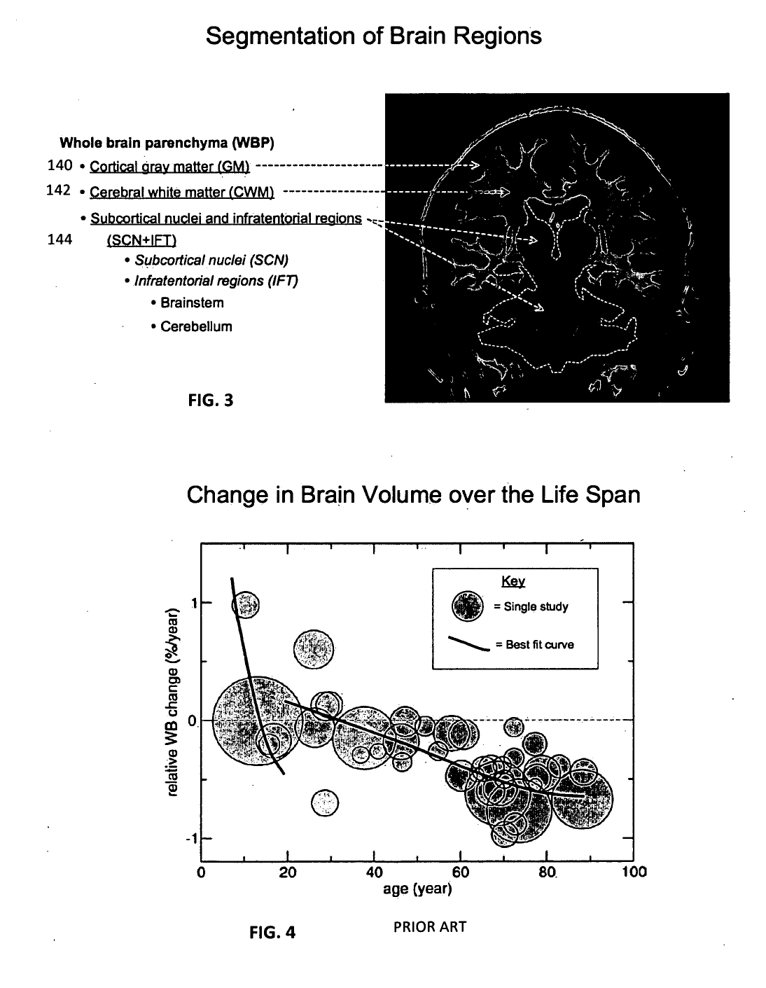

By way of example, and not limiting in nature, FIG. 3 illustrates a sample data scan of a patient, where this exemplary scan data is from an MRI scan. The scan illustrates the whole brain parenchyma (WBP) is divided into 3 sections. A first section 140 is the cortical gray matter (GM). A second section 142 is the cerebral white matter (CWM). The third section in FIG. 3 is the subcortical nuclei (SCN) and infratentorial (IFT) regions 144. The IFT region is further subdivided into the brainstem and cerebellum regions, not expressly illustrated in FIG. 3.

This approach follows the lead of Hedman, who analyzed brain growth/atrophy data for 3 brain regions: 1) WBP; 2) GM; and 3) CWM. The remaining brain parenchyma consists of SCN and IFT (collectively referred to as “SCN+IFT”) and can be deduced by subtracting GM and CWM from WBP.

In one embodiment, the volume of the WBP is determined based on the summation of the values of the GM with the CWM with the SCN+IFT.

WBP=GM+CWM+(SCN+IFT) [Equation 1]

The currently proposed method has been applied to WBP, GM, CWM and SCN+IFT. It can be applied to any brain subregion. In the current application, in most cases, for the sake of simplicity, we will refer simply to “brain volume,” which can be applied to whole brain or any brain subregions.

Whereas these regions grow and shrink over the span of the patient's life, a brain injury event can cause a dramatic or abnormal change in one or more of these regions.

With respect to FIG. 2, a next step, step 122, is electronically calculating a current brain volume of the patient based on the current intracranial volume data. The processing device 102 of FIG. 1, based on executable instructions 104, can perform this step.

In one embodiment, the brain scan data is processed to acquire data values usable for brain volume calculations. In one embodiment, the brain scan data is processed by NeuroQuant®, available from CorTech Labs, Inc., San Diego, Calif., to measure intracranial volume. It is recognized that any other suitable brain volume software calculation and processing techniques and/or software may be utilized.

The scan data is processed to generate the data values used to calculate the current brain volume.

In one embodiment, the brain volume of the WBP is determined based on the summation of the values of the GM with the CWM with the SCN and IFT.

The next step in the method of FIG. 2, step 124, is electronically calculating/estimating a pre-injury brain volume based at least on the current intracranial volume data and the age of the patient at the time of the injury. Again with reference to FIG. 1, the processing device 102 performs processing operations in response to the executable instructions 104 to calculate the prior brain volume.

Another application of the brain volume estimation method is to age-adjust normal control data. Since age has important effects on brain volume, in general, it is desirable to match age between patients and normal control subjects. If the age of the normal controls differ from that of the patient, a less desirable but still useful option would be to age-adjust the normal control data using the brain volume estimation method.

Using the methodology described herein, the brain volume of each normal control can be age-adjusted to match that of the individual patient or the group of patients. This approach is analogous to the traditional approach of co-varying out age differences between groups. But the age-adjustment method based on the volume estimation model is better than that of the traditional covariance approach because the former has a priori information about normal brain volume, in contrast to the latter. In other words, the volume estimation model is based on 56 studies in normal controls (2,211 normal subjects) throughout the life span; in contrast, the covariance approach usually would be limited to much smaller samples and much more limited age spans.

Although the general consensus is that intracranial volume does not change during adult life, it is possible that it may decrease slightly during old age, e.g. in the 70s or older. Previous studies have not found that, but in the future, if larger studies were done, it is possible that small but significant changes in intracranial volume could be found in old age. If so, normal growth/atrophy curves for intracranial volume could be developed, similar to Hedman's growth curves for brain volume. The growth/curves for intracranial volume could be integrated into the brain volume estimation model.

Prior techniques for calculating brain volume, e.g. Hedman, were based on the non-occurrence of a brain injury event, that is, they were based only on normal subjects. The calculating of step 124 improves upon Hedman. Without a brain injury, i.e. in normal subjects, brain volume changes in a predictable way across the life span, visible in FIG. 4, the Hedman brain growth/atrophy curve.

Where Hedman maps brain growth/atrophy across a lifespan, the processing operations of step 124 further include an offset factor accounting for the brain injury event.

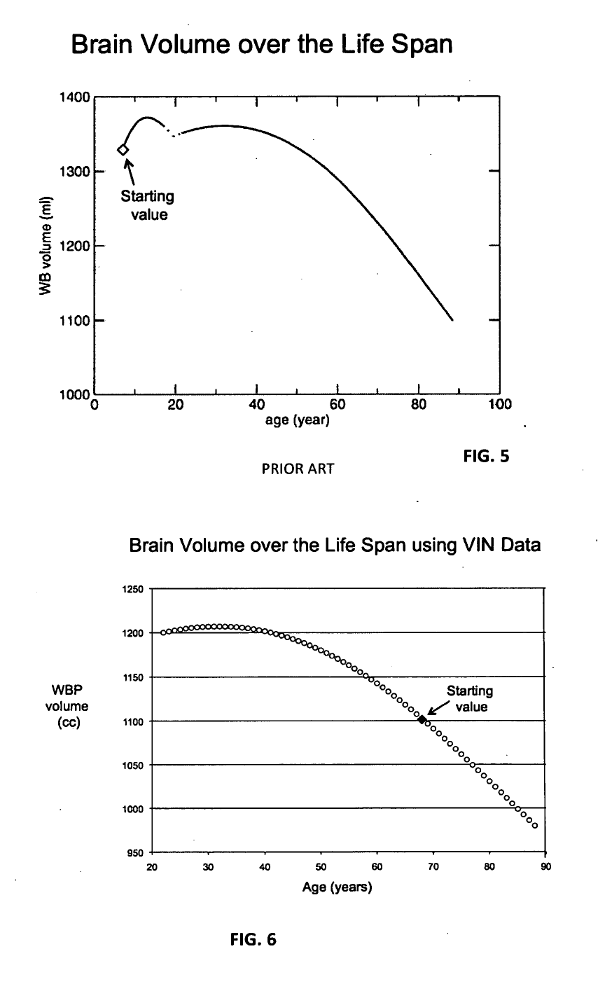

FIG. 5 is taken from Hedman. Hedman used the growth/atrophy curves shown in FIG. 4 to plot whole brain volume versus age across the lifespan. They began by entering starting data (indicated by the diamond) into the model using the mean whole brain volume (measured in cc) in 9 year old normal subjects, using data from Peper et al (2009): Heritability of regional and global brain structure at the onset of puberty: A magnetic resonance imaging study in 9-year-oldtwin pairs. Hum Brain Map 30:2184-2196 (“Peper's”) study of 9 year old twins; N=210. Then they used the growth/atrophy curves of FIG. 4 to estimate/calculate by integration whole brain volume at other points in the lifespan.

FIG. 6 illustrates the application of the Hedman method to brain volume data from our own normal subjects. It shows a graphical representation of plotting estimated whole brain parenchymal (WBP) volume across the life span. The estimation method was very similar to Hedman, FIG. 5. Using the starting point, the model calculated (by integration) WBP volume across the life span. Similar to Hedman's method, the starting value was actually measured, not simply estimated: the mean of WBP volume for our sample of 20 normal control subjects was 1102 cc at the mean age of 68 years.

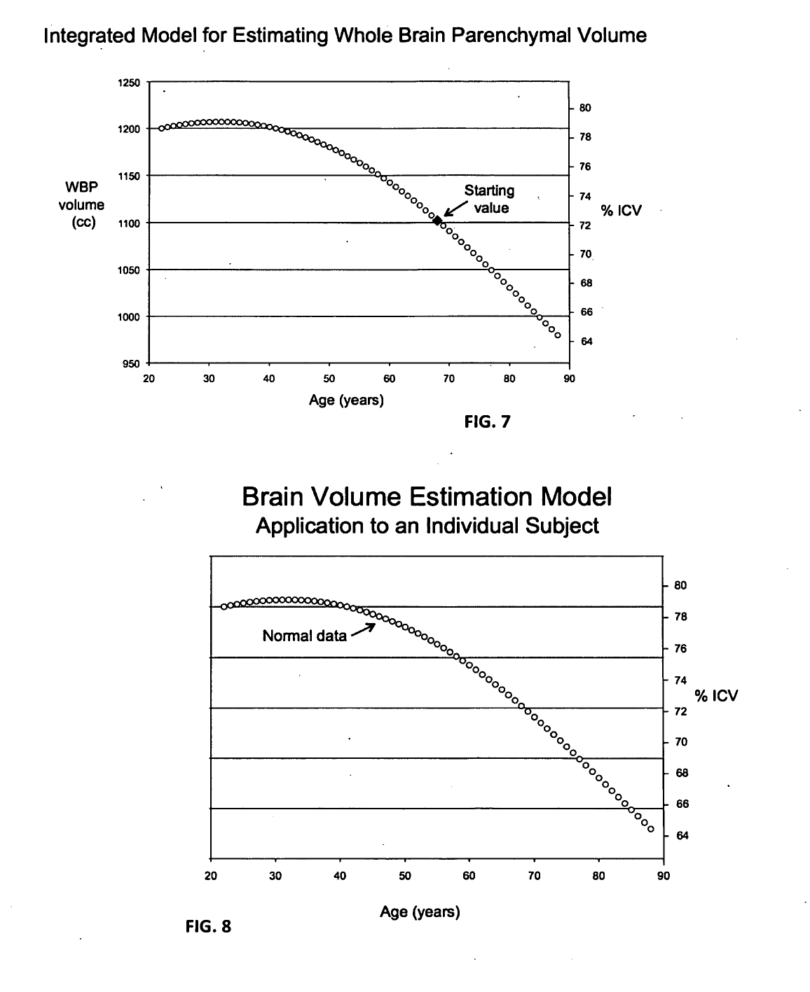

FIG. 7 is similar to FIG. 6, except that it further includes a right hand margin to map brain volume measured as a percentage of intracranial volume (% ICV) onto WBP volume measured in cc. This step enabled using post-injury ICV to estimate pre-injury brain volume circumventing Hedman's limitations. In FIG. 7, the starting value for WBP volume of 1102 cc is mapped on to the % ICV of 72.2%, and accordingly the mean ICV of this sample was 1527 cc. More generally, for any normal subject who has an ICV of 1527 cc, the graph of FIG. 7 provides for estimating their brain volume throughout the lifespan.

FIG. 8 is similar to FIG. 7, except that the left-hand axis (WBP volume measured in cc) was removed. The resulting graph is applicable to any normal subject with any intracranial volume. For example, given a specific age, a normal subject's brain volume measured as % ICV can be estimated at that age.

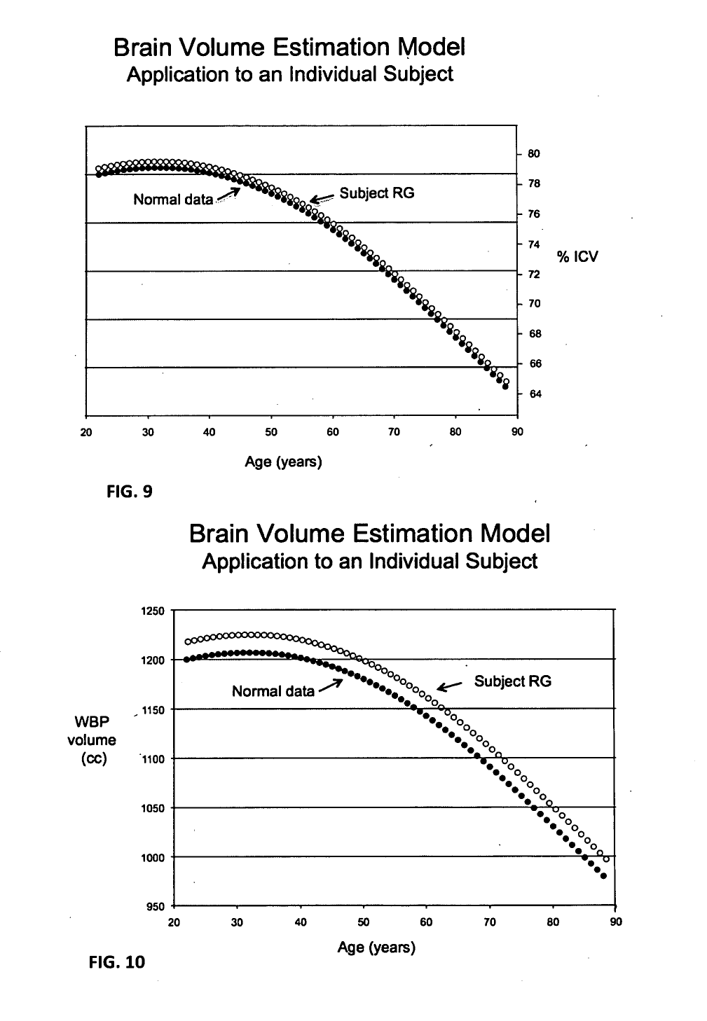

FIG. 9 is similar to FIG. 8, except that a single subject's expected data are overlaid on the data derived from the sample of 20 normal controls. For purposes of visibility, the Subject RG line is slightly offset, but under proper computations, the subject's data is actually an exact overlay of the group data.

FIG. 10 is similar to FIG. 9, except that the right-hand axis (WBP volume measured as % ICV) was removed, and the left-hand axis (WBP volume measured in cc) was replaced. As a result of these changes, Subject RG's curve was displaced upward relative to the normal control data, because his intracranial volume (1546 cc) was bigger than the mean of our normal control group (1527 cc). In other words, since his skull was bigger, the model predicted that his brain would be bigger throughout his life span. More generally, for any normal subject who has an ICV of 1527 cc, the data corresponding to the graph of subject RG's data provide a method for estimating their brain volume (cc) throughout the lifespan.

FIG. 11 shows the results of a test of the reliability of the brain volume estimation method. It is a graph of measured whole brain parenchymal (WBP) volume vs. estimated WBP volume for the normal control subjects. In this embodiment, since the ages of the normal control subjects varied from 60 to 72 years, differing from the mean age of 68 years, it provided an opportunity to test the reliability of the volume estimation method. Using the method described above, WBP volume was estimated for each normal control based on ICV. The estimated values correlated highly with measured values of WBP volume (ICC=0.95), supporting the reliability of the model. Similarly, high reliabilities were found for GM (ICC=0.81) and CWM (ICC=0.89).

Although the primary application of the proposed brain volume estimation method is for patients with brain disease or injury (e.g. TBI patients) it also is useful in normal control subjects. The example above shows one application, i.e. for testing reliability of the method. Another application in normal subjects is for controlled research studies, e.g. so that the same method that is applied to patients with brain injury also is applied to normal control subjects.

FIG. 11 shows the application of the brain volume estimation method to a patient with brain injury. Patient RG was injured at age 30; before then, he was normal. Therefore, estimates of his brain volume can be made reliably up to age 30 using ICV measured later in life. His estimated brain volume before injury can then be compared to his brain volume measured after injury. The change of brain volume over time can be compared to that of normal control subjects.

In one embodiment, the processing device 102 of FIG. 1 may utilize a spreadsheet or other type of software interface for determining t0 brain volume, the brain volume estimation prior to brain injury event. The spreadsheet application utilizes the intracranial volume and age of the subject to generate the estimated brain volume.

In one embodiment, the processing device 102 of FIG. 1 may utilize a spreadsheet or other type of software interface for determining t0 brain volume, the estimated pre-injury brain volume. The spreadsheet application utilizes the intracranial volume (measured after injury) and age of the patient just before the injury to generate the estimated pre-injury brain volume.

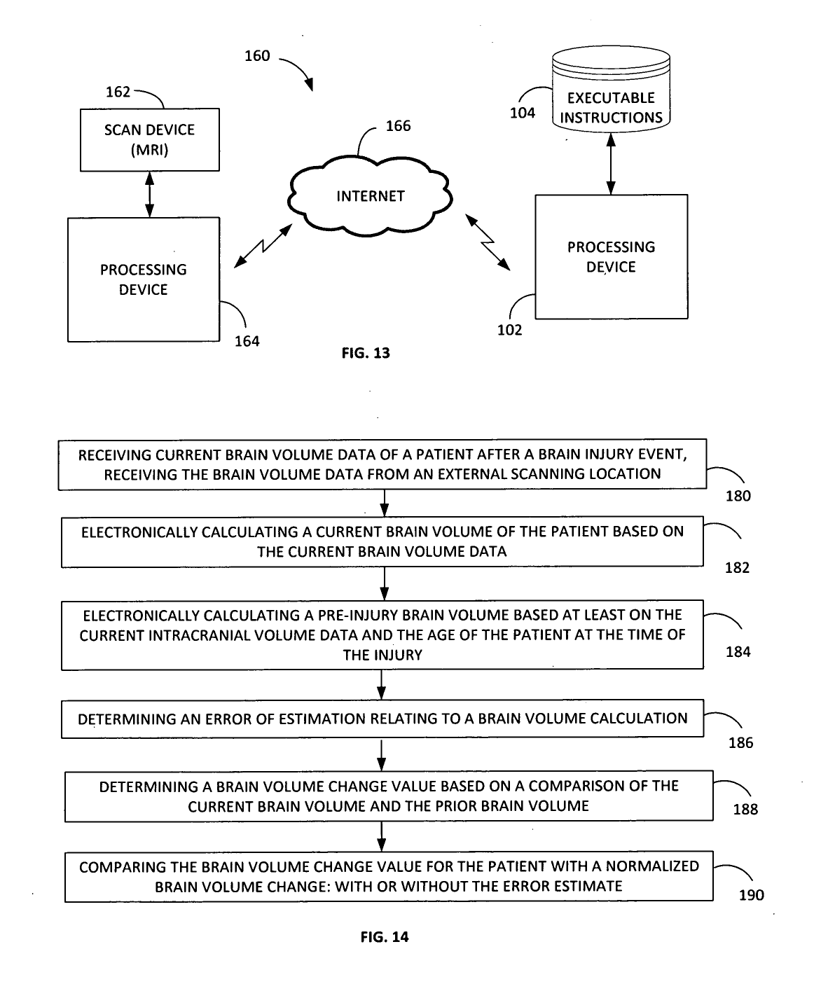

FIG. 13 illustrates another embodiment of a system for calculating a brain volume for a patient having a brain injury event. Similar to FIG. 1, the system of FIG. 13 includes the processing device 102 and computer readable medium having executable instructions 104 stored therein. The system further includes a scan device 162, a processing device 164 associated with the scan device and a network 166.

The scan device 162 may be any suitable device operative to take a scan or measurements of a patient's brain, including a MRI device. The device 162 may be located distant from the processing device 102, such being housed at a radiology department in a hospital, at an imaging clinic, or any other suitable location.

The processing device 164 includes processing functionality for receiving and processing the scan data from the device 162, wherein the processing device 164 operates in manners known to those skilled in the art for processing the scan data from the device 162 and making said data available to other processing systems. Such functionality may include storage and/or transmission of scan data to other processing systems.

The network 166 may be any suitable network allowing for communication thereacross. In a typical embodiment, the network 166 is the Internet, but can be any other type of network including a private intranet for communicating to the processing device 102.

The devices 102 and 164 include communication techniques as recognized by one skilled in the art for communicating and sharing scan data. The communication may include security or transmission optimization techniques, such as relating to electronic medical records.

Wherein, the processing device 102 operates similar to the operations described above with respect to FIG. 1, including executable instructions for calculating a brain volume. In the embodiment of FIG. 13, the scan data is acquired from an off-site or third party scan service. The calculation of the brain volume estimation uses this third-party data, whereby the method and system does not, in this embodiment, require the acquisition of the scan data to be concurrent in time or geography with the measurement determination. It is noted that further embodiments may include the scan data acquisition being concurrent in time and/or place with the calculation such that the embodiment of FIG. 13 is not limiting in nature.

FIG. 14 illustrates a flowchart of the steps of one embodiment of a method for calculating/estimating brain volume of a brain injury patient. The method steps may be performed electronically using one or more processing devices, such as the processing device 102 of FIGS. 1 and 13.

A first step, step 180, is receiving the current brain volume data of a patient after a brain injury event. In this step, the brain volume data is received from an external scanning location, such as scanning device 162 (e.g. MRI scanner) of FIG. 13.

A next step, step 182, is electronically calculating a current brain volume of the patient based on the current brain volume data. Similar to step 122 of FIG. 2, this step may be performed using known brain volume calculation techniques, including brain volume calculation software as available.

Step 184 provides for electronically calculating a pre-injury brain volume based at least on the current intracranial volume data and the age of the patient just before the injury, the prior brain volume being at a point in time prior to the brain injury event. This step may be performed electronically is described above, wherein the calculations may include the error estimate for determining a brain volume range.

Step 186 of the method provides for determining the error of estimation (analogous to error of measurement) relating to a brain volume calculation. Whereas calculations of a brain volume prior to a brain injury event are estimated values, such values generally will be somewhat higher or lower than actual brain volume, if it could have been measured. Using normal control data, the estimation error can be determined, and this was done for our pilot study. These error estimation values fall within a mathematical range, including a mean and a normal (bell-shaped) distribution curve. Within the normal curve, estimation error can be determined based on the standard deviation of the distribution. The estimation error can be used to determine a range of confidence within the distribution, typically indicated as a range of percentile ranks, e.g. from the lower 5th percentile to the upper 5th (i.e. 95th) percentile. Accordingly, the output of the brain volume estimation model, i.e. the estimate of the patient's brain volume before the injury, would be the best estimate possible, but there would be a 90% chance that the actual brain volume, if measured, would have been found to lie between the 5th and 95th percentiles of the error estimation distribution, with the best estimate set as the mean of the distribution. In some settings (e.g. legal) it may be useful to choose an estimate of brain volume that is particularly conservative and has a greater chance of minimizing false positive findings. For example, if one were testing the idea that brain volume decreased from before to after an injury, one might choose a pre-injury estimate of brain volume that fell at the lower 5th percentile, making it very unlikely that one was overestimating pre-injury brain volume. This can be called the “conservative estimate.” If the patient's brain volume decreased significantly from before to after injury, using the conservative estimate, then one would be more confident that a positive finding was due simply to estimation error.

Step 188 includes determining a brain volume change based on a comparison of the current brain volume and the estimated prior brain volume. This step may be performed by electronically comparing brain volume measurements at these time points for determining the delta or change in values across the time period.

Step 190 provides comparing the patient's change in brain volume to that of a normal control group. The comparison of step 190 may be performed with or without use of the estimation error, i.e. best estimate or conservative estimate of pre-injury volume may be used.

Usually percentage change in brain volume over time is calculated as follows:

(1) Determine change in brain volume by subtracting volume at t0 from volume at t1. Equation: (Brain volume change)=(volume at t1)−(volume at t0).

(2) Express the result as a percentage. Equation: (Brain volume % change)=(Brain volume change)×100%.

(3) Divide brain volume % change by change in time. Equation: (Brain volume % change over time)=(Brain volume % change)/(t1−t0). Commonly, time is measured in years, in which case this step annualizes the volume change data.

Although brain volume change data usually are annualized, the annualization of data causes a problem for data collected less than one year apart. For example, in our pilot data, the normal control brain volume data were collected one year apart, and the volume change data were annualized. Similarly, for patients who had brain volume data collected one or more years apart, their volume change data were annualized without problem. However, for patients who had brain volume data collected less than one year apart, annualizing the data caused amplification of measurement error. For example, consider change of whole brain volume. Whole brain volume in normal young to middle-aged adults decreases on average approximately 0% per year, with the normal range extending from −2% per year at the 5th percentile, and up to 2% per year at the 95th percentile. To take an extreme example for explanatory purposes, if a patient's whole brain volume decreased 1% in a week, that would be well within the range of measurement error. But if those data were annualized, the results would suggest that his brain atrophied 52% in a year, an extremely rapid rate that probably would be fatal and would not make biological sense. Therefore, one cannot annualize data collected less than one year apart because it may amplify measurement error.

A partial solution to the limitation of not being able to annualize data collected less than one year apart is simply to use non-annualized data. Following the above example, if a patient's brain atrophied 3% in 6 months, the non-annualized rate of atrophy could be compared to the normals' annualized rate of atrophy. In this example, since the patient's brain shrank more in 6 months than normal brains would be expected to shrink in 1 year, we can be confident that the patient's brain shrank abnormally fast.

Pilot Study:

A pilot study was performed comparing twenty six patients with mild or moderate TBI to twenty normal control subjects. Software processing measured MRI brain and intracranial volumes, where brain volumes after an injury, at times t1 (the time of the first MRI) and t2 (the time of the second MRI), were compared with brain volumes just prior to the injury, volume estimated at t0, using longitudinal designs. Groups were compared with respect to volume changes in the WBP and three major subdivisions: GM; CWM; and SCN+IFT.

Pilot Study: Main Results

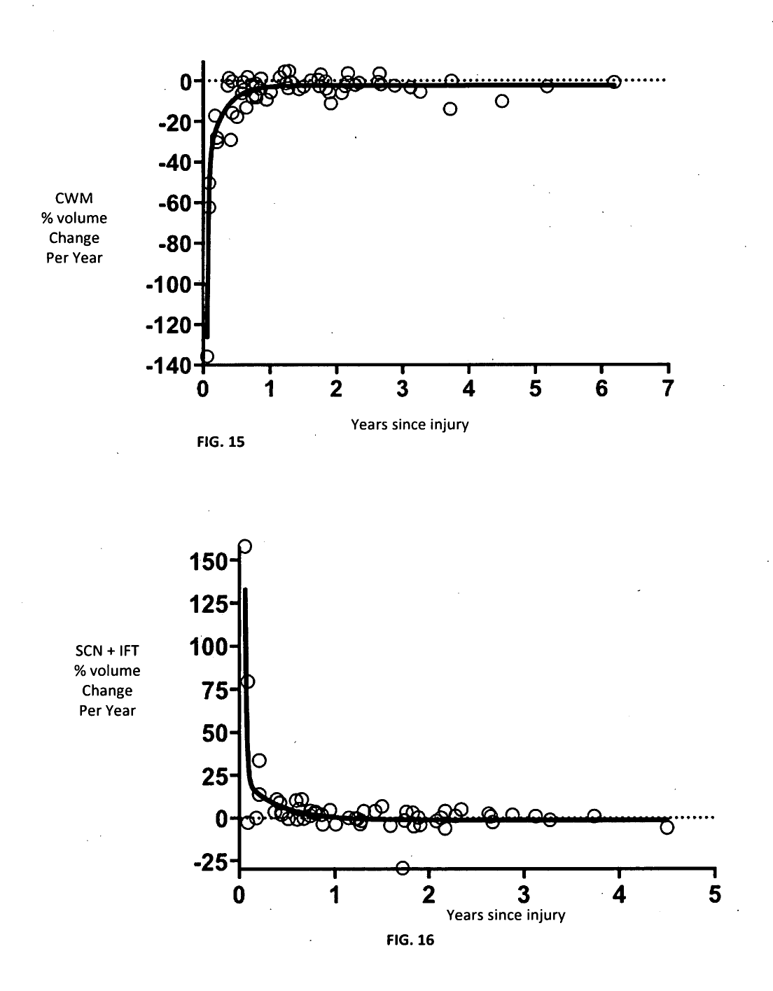

In the pilot study using the volume estimation method, changes in brain volume before (at t0) and after injury (at t1) were compared to changes over time in the normal control group. During the initial phase after injury (t0 to t1), the TBI patients had abnormally rapid atrophy of WBP and CWM (FIG. 15) and abnormally rapid enlargement of SCN+IFT (FIG. 16). Rates of volume change during t0-t1 correlated with cross-sectional (i.e. single point in time) measures of volume abnormalities at t1, supporting the internal reliability of the volume. A logistic regression analysis using the volume change data produced a function that perfectly predicted group membership (TBI patients vs. normal control subjects), supporting an important practical application of the method, i.e. diagnosis of TBI.

FIG. 15 illustrates CWM volume change per year versus time after injury. This graph shows that patients with brain injury events had extremely rapid atrophy of CWM close to the time of injury. Each point represents a patient's volume change data at the midpoint of the t0-t1, t1-t2 or t2-t3 time interval (where t0=time of injury; t1=time of first MRI after injury; t2=time of second MRI after injury; and t3=time of third MRI after injury). Fifty-nine data points were available for 26 patients. Curve fitting techniques showed a statistically significant fit for a 2-phase association exponential curve (solid curve).

FIG. 16 illustrates a curve of SCN+IFT volume change per year versus time after injury. This graph shows that patient with brain injury events had extremely rapid enlargement of SCN+IFT close to the time of injury. Each point represents a patient's volume change data at the midpoint of the t0-t1, t1-t2 or t2-t3 time interval. Fifty-six data points were available for 26 patients. Curve fitting techniques showed a statistically significant fit for a 2-phase association exponential curve (solid curve).

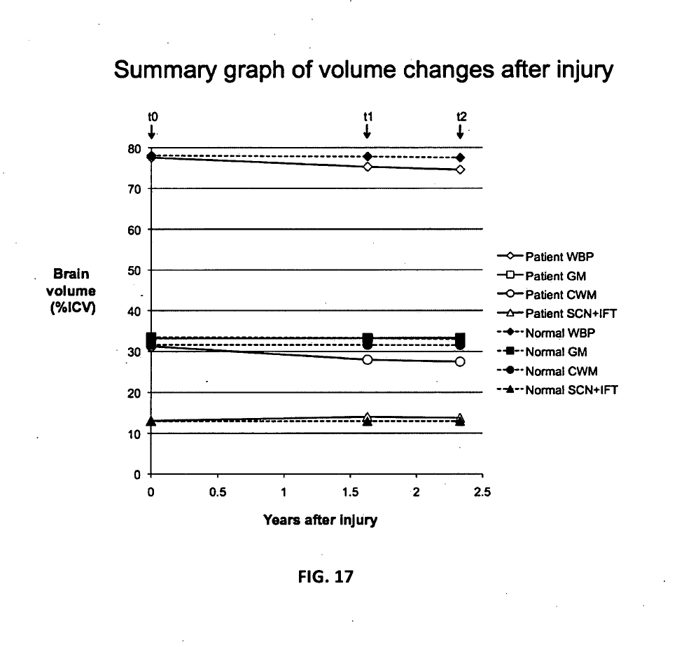

FIG. 17 summarizes graphically the results of our pilot study. Brain volume changes in TBI patients are compared to brain volume changes in normal control subjects. Each of the normal control subjects had 2 MRIs performed 1 year apart. The age-adjustment method (see above) were used to age-match these data to those of the patients. The graph shows that patients had significant atrophy of WBP and CWM from t0 to t1 to t2. SCN+IFT enlarged rapidly from t0 to t1, then decreased somewhat from t1 to t2. It is noted that in this embodiment, the GM changed little and nonsignificantly. The unique pattern of changes, with some regions shrinking and some regions enlarging, allowed for the logistic regression model (see above) to perfectly separate the groups.

It is recognized that additional factors may be utilized in estimating brain volume, including the patient's medical history, family history, environmental factors, education levels, etc. Other demographic or measurable variables which similarly might correlate with brain volume can include: IQ or scores on scholastic aptitude tests; level of function at the job; occupation; income; sex; race; and measures obtained from other brain/head scans that were not used in the primary analyses upon which the volume estimation was based (e.g. the pilot study used MRIs which allowed NeuroQuant analysis; it is possible that CT scans or MRIs which do not allow NeuroQuant analysis could be used to measure intracranial volume and could therefore provide useful data for the volume estimation method).

The reliable determination of the effects of the brain injury thus allows numerous uses to practitioners. The change in brain volume based on the brain injury can be used to help prescribe treatments for the patient, help assist in the diagnosis of brain injuries for the patient as well as provide determinations of region-specific changes. In another embodiment, the change in brain volume can be used to help assess the change in the patient's capacity or quality of life after the brain injury event, relative to the person's state of being prior to the brain injury event.

Prior techniques required the actual measurement of brain volume prior to injury or illness, which is impractical in most situations. The brain volume estimation can be applied to any subject who was normal before the time of injury or disease onset, or who had disorders beforehand, which did not affect the measurement of brain volume. This would include the majority of the general population.

More generally, in the areas of neuropsychiatry and general psychiatry, there is a great need for objective diagnostic tests. Decades of research and clinical work with brain imaging techniques have provided excellent diagnostic techniques for general neurological disorders (for example, stroke and brain tumors) but little to no diagnostic techniques for neuropsychiatric or general psychiatric disorders, in which the effects on brain volume are subtler. The brain volume estimation technique probably can be used as a diagnostic test for mild or moderate TBI and may prove to be useful in the diagnosis of other brain disorders.

The present method and system is additionally applicable to matters outside of human patients. The present methodology is applicable to any patient having a skull and associated cranial volume. For example, the technique is applicable to animals, such as dogs or rats, and determining intracranial volume across a range in time.

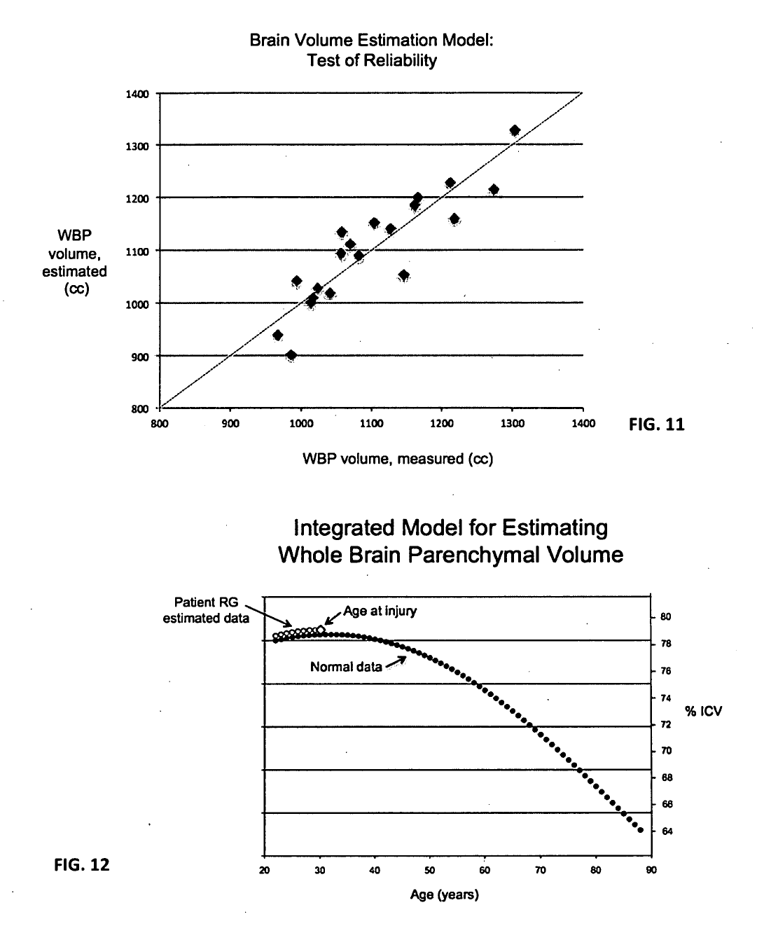

FIGS. 1 through 17 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, Applicant does not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments so fully reveals the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein.

What is claimed is:

1. A computerized method for calculating brain volume comprising:
receiving current intracranial volume data of a patient;
electronically calculating a current brain volume of the patient based on the current brain volume data; and
electronically calculating a prior brain volume based at least on the current intracranial volume data and an age of the patient at the time of the brain injury.

2. The method of claim 1, wherein the current intracranial volume data of the patient is after the brain injury, the method further comprising:
electronically calculating the prior brain volume at a point in time prior to the brain injury.

3. The method of claim 2 further comprising:
comparing a brain volume change value for the patient with brain volume change data for a group of normal control subjects.

4. The method of claim 2 further comprising:
for patients with brain volume data obtained at least one year apart, annualizing a brain volume change over a period of time between a current brain volume and a prior brain volume;
calculating a brain volume change value based at least in part on the annualized brain volume change, and
comparing the patient's annualized brain volume change to a normal controls' annualized brain volume change.

5. The method of claim 2 further comprising:
for patients with brain volume data obtained less than one year apart, determining a non-annualized brain volume change between the current brain volume and the prior brain volume;
calculating the brain volume change value based at least in part on the non-annualized brain volume change, and
comparing the patient's non-annualized brain volume change to a normal control group's annualized brain volume change.

6. The method of claim 1 further comprising:
determining an estimation error relating to the calculating the brain volume; and
applying the estimation error to the prior brain volume, thereby allowing a choice between using a best estimate or a conservative estimate of pre-injury brain volume.

7. The method of claim 1, wherein the brain volume relates to at least one of: whole brain parenchyma, cortical gray matter, cerebral white matter, and subcortical nuclei and infratentorial regions.

8. The method of claim 1, wherein the current brain volume data is acquired from a magnetic resonance imaging (MRI) scan.

9. The method of claim 7, wherein the current brain volume is electronically received from an MRI scan performed by a third party service provider.

10. The method of claim 1, wherein the brain injury is at least one of: a traumatic brain injury and a brain malady.

11. The method of claim 1, wherein the patient is at least one of: a patient having a brain injury and a normal subject.

12. An apparatus for calculating brain volume comprising:
at least one processing device; and
executable instructions stored in a computer readable medium such that the processing device, in response to the executable instructions, is operative to:
receive current intracranial volume data of a patient;
electronically calculate a current brain volume of the patient based on the current brain volume data; and
electronically calculate a prior brain volume based at least on the current intracranial volume data and an age of the patient at the time of the brain injury.

13. The apparatus of claim 12, wherein the current intracranial volume data of the patient is after a brain injury event, the processing device further operative to:
electronically calculate the prior brain volume at a point in time prior to the brain injury.

14. The apparatus of claim 13, the processing device further operative to:
compare a brain volume change value for the patient with brain volume change data for a group of normal control subjects.

15. The apparatus of claim 13, the processing device further operative to
for patients with brain volume data obtained at least one year apart, annualize a brain volume change over a period of time between a current brain volume and a prior brain volume;
calculate a brain volume change value based at least in part on the annualized brain volume change, and
compare the patient's annualized brain volume change to a normal control group's annualized brain volume change.

16. The apparatus of claim 13, the processing device further operative to:
for patients with brain volume data obtained less than one year apart, determine a non-annualized brain volume change between the current brain volume and the prior brain volume;
calculate the brain volume change value based at least in part on the non-annualized brain volume change, and
compare the patient's non-annualized brain volume change to a normal control group's annualized brain volume change.

17. The apparatus of claim 13 further comprising:
determine an estimation error relating to the calculating the brain volume; and
apply the estimation error to the prior brain volume, thereby allowing a choice between using a best estimate or a conservative estimate of pre-injury brain volume.

18. The apparatus of claim 12, wherein the brain volume relates to at least one of: whole brain parenchyma, cortical gray matter, cerebral white matter, and subcortical nuclei and infratentorial regions.

19. The apparatus of claim 12, wherein the current brain volume data is acquired from a magnetic resonance imaging (MRI) scan.

20. The apparatus of claim 12, wherein the brain injury is at least one of: a traumatic brain injury and a brain malady.
